(19) United States
(12) Patent Application Publication
(10) Pub. No.: US 2002/0042584 A1
(43) Pub. Date: Apr. 11, 2002

(54) Flexible back brace

(76) Inventor: Michael W. Rue, Fletcher, NC (US)

Correspondence Address: HAHN LOESER & PARKS, LLP, TWIN OAKS ESTATE, 1225 W. MARKET STREET, AKRON, OH, 44313, US

(21) Appl. No.: 09/900,747

(22) Filed: Jul. 6, 2001

Related U.S. Application Data
(60) Provisional application No. 60/216,459, filed on Jul. 6, 2000.

(51) Int. Cl. A61F 5/00

(52) U.S. Cl. 602/5

(57) ABSTRACT

This invention provides a device which restrains a user from undesirable movement while permitting desirable movement when the user picks up an object from a floor or the ground. This device is a flexible and adjustable back brace which, when worn by a user, restrains the user from excessive bending over at the waist to pick up an object, while permitting the user to pick up such object by bending at the knees so that the user's back remains straight. The device is adjustable to fit users of different sizes.

### TECHNICAL FIELD

This invention relates to a flexible back brace which is capable of restraining a user from bending over at the waist in order to pick up an object.

### SUMMARY OF THE INVENTION

This invention provides a device which restrains a user from undesirable movement but permitting desirable movement when the user picks up an object from a floor or the ground. This device is a flexible and adjustable back brace which, when worn by a user, restrains the user from bending over at the waist to pick up an object while permitting the user to pick up such object by bending at the knees so that the user's back remains straight. The device is adjustable to fit users of different sizes.

### BRIEF DESCRIPTION OF THE DRAWINGS

In the drawings:

FIG. 1 is a front elevational view of the back brace of this invention, shown with shoulder and leg straps unfastened.

FIG. 2 is a fragmentary side elevational view of a multiple ply elastic web which forms part of the back brace of this invention.

FIG. 3 is an exploded view of a releasable fastener used to secure shoulder straps or leg straps together.

FIG. 4 is an elevational view of the fastener of FIG. 3 in closed or assembled condition.

FIG. 5 is an elevational view of a clamp for adjustably securing upper and lower portions of the back brace of this invention together.

FIG. 6 is a front view of a user who is wearing a back brace according to this invention.

FIG. 7 is a front view of a user who is wearing a back brace according to this invention.

### DESCRIPTION OF THE PREFERRED EMBODIMENT

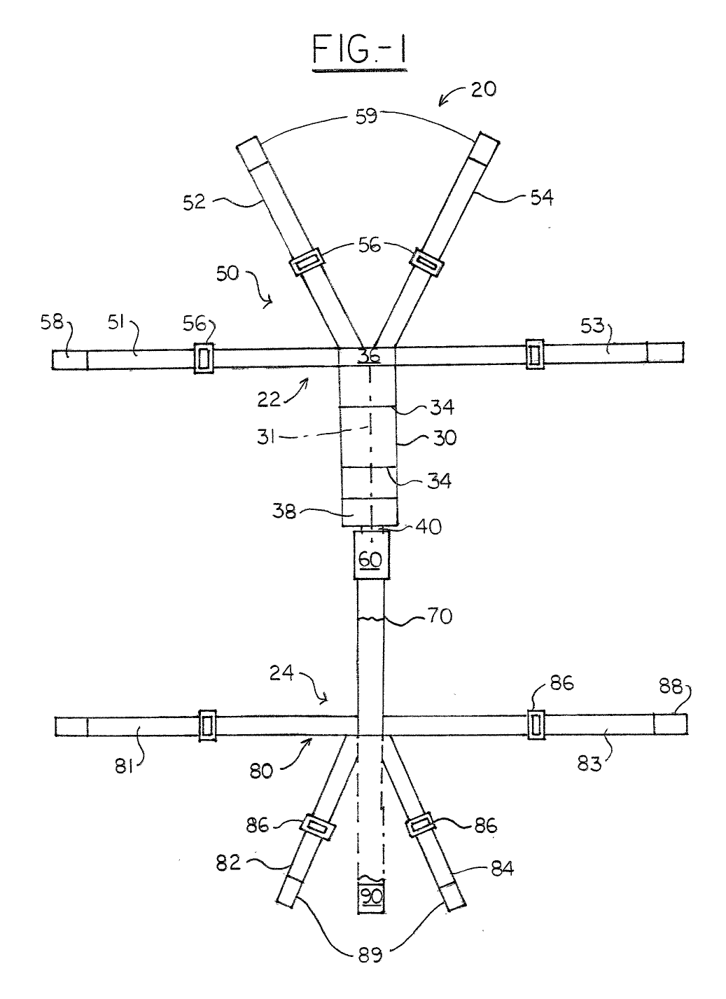

Referring now to FIG. 1, the device of this invention as a whole is a flexible and adjustable back brace 20. Back brace 20 comprises an upper portion 22 and a lower portion 24, which are adjustably secured together.

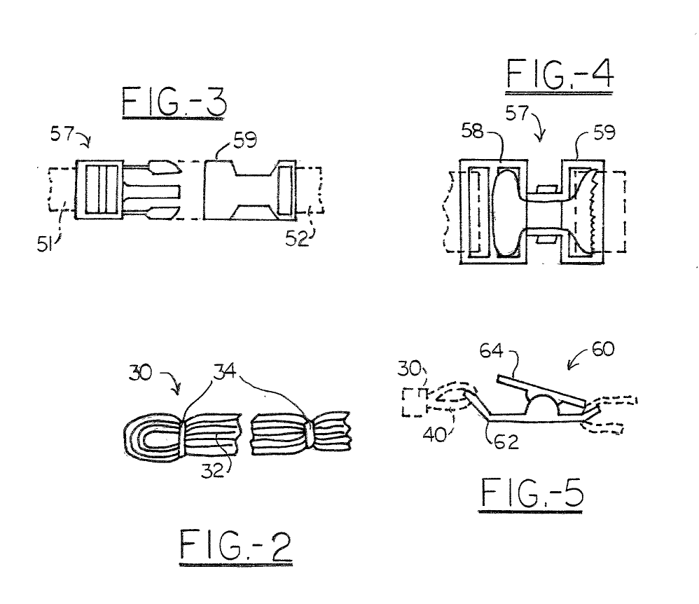

The upper portion 22 of device 20 comprises a longitudinally extending and preferably rectangular elastic multiple ply center web (or upper torso web) 30. Center web 30 has a longitudinal axis or center line 31 and is symmetrical with respect to axis 31. Center web 30 is formed of a plurality of plies 32 (six are shown) of elastic cloth, as may be seen in FIG. 2. Preferred plies 32 of elastic cloth are woven and have roughened surfaces. The plies 32 are sewn together by transverse stitching 34 at longitudinally spaced locations to form a unitary structure.

Use of elastic material to form center web 30 permits some longitudinal stretching. The modulus (or stress-strain ratio) of web 30 is determined by the number, width and stiffness of plies 32 which form web 30. Elastic cloth is preferable to inelastic or dimensionally stable cloth so that the device 20 will have some “give” when a wearer or user tries to bend his or her back. However, the device offers appreciable resistance so that a user can bend his or her back only to a limited extent. This protects the user from back strain and injury.

A first part 36 of a two-part closure member is sewn to upper torso web 30 at the first or upper end thereof. The purpose of this closure member will become apparent when the second part is described subsequently. The closure member is preferably cloth. The preferred closure member is a hook-and-loop closure member (e.g., “Velcro”), and the first part 36 may be the loops of such closure member.

A cloth reinforcing member 38, preferably rectangular, may be sewn to the center web 30 at its second or lower end for reinforcement and to prevent fraying. A cloth belt loop 40 may be sewn to the center web 30 at its lower end to receive a clamp or another connector (to be described later) which serves as a connector between the upper portion 22 and the lower portion 24 of center web 30.

A shoulder harness 50, comprising four adjustable cloth straps 51, 52, 53 and 54, is sewn to the first or upper end of center web 30. Each strap is sewn at its first or inner end to the center web 30. These straps may be of conventional woven cloth belting material (e.g., nylon) having a rough surface. The straps are non-stretchable (i.e., dimensionally stable or inextensible) and are much narrower than center web 30. Straps 51, 52, 53 and 54 are adjustable in length (to fit users of different sizes). A loop is formed at an outer end of each of the straps 51, 52, 53 and 54. Each of these straps is provided with a buckle 56, which may be conventional, for adjustment or length. Length may be adjusted in a conventional manner.

Straps 51 and 52, when fastened together, form a first pair which may encircle a user's left shoulder. Similarly, straps 53 and 54 may be fastened together to form a second pair which may encircle a user's right shoulder. A fastener 57 (see FIGS. 3 and 4) is provided for this purpose. Each fastener 57 comprises two complementary members, e.g., a male member 58 and a female member 59, which may be releasably fastened to each other. Male members 58 may be received in outer end loops of straps 51 and 53, and female members 59 may be received in outer end loops of straps 52 and 54.

Fastener 57 may be a quick release fastener. A preferred fastener is a side lock fastener, which is known in the art and is shown in FIGS. 3 (unfastened) and 4 (fastened). This fastener 57 comprises a three-pronged male member 58 which is adapted to be releasably received in a female member or receptacle 59. Fastener 57 locks when fastened, as shown in FIG. 4. To release, one presses the two outer prongs of male member 58 inwardly.

In an alternate embodiment, each of these straps 51, 52, 53, and 54 are formed with hook and loop material, such as VELCRO brand hook and loop, on the ends thereof for fastening the straps together. By using hook and loop material, the user is able to adjust the fit of the straps to the user's upper-body more easily and is able to wear the system 20 with greater comfort. Further, the hook and look-fastening material used for straps 51-54 also eliminates the need for fastener 57.

Straps 51 and 53 are preferably disposed at right angles to axis 31. Straps 52 and 54 are preferably disposed at an acute angle, say 30° (which is not critical) to axis 31.

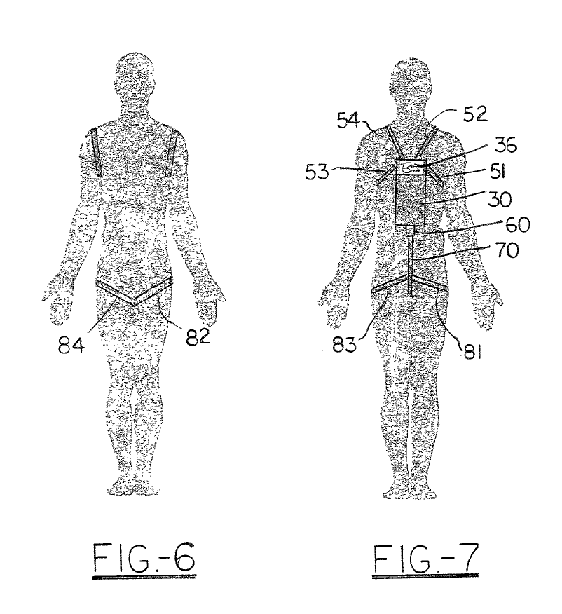

A clamp 60 (FIG. 5) adjustably connects upper portion 22 and lower portion 24 back brace 20. Clamp 60 includes a base member 62 having mounted thereon a pivotally mounted clamping member 64, which is spring biased toward closed or clamping position.

The lower portion 24 of back brace 20 comprises a longitudinally extending, non-stretchable strap 70. Strap 70 is a lower torso strap which permits adjustment of device 20 to fit users of different heights. Strap 70 may be of a woven cloth material (e.g., nylon) having a roughened surface. When the device 20 is in use (see FIGS. 6 and 7), strap 70 hangs vertically below center web 30 in axial alignment therewith. Strap 70 is preferably much narrower than center web 30.

Attached (e.g., by sewing) to the lower (or first) end of longitudinally extending strap 70 is a leg harness 80 which comprises four adjustable leg straps 81, 82, 83 and 84. Each is provided with a buckle 86 for length adjustment. Straps 81 and 83 extend at right angles to axis 31 of strap 70, and straps 82 and 84 extend at acute angles (e.g., 30°) to axis 31 of strap 70. Straps 81 and 83 extend from strap 70 to male fastener members 88, and straps 82 and 84 extend to female fastener members 89. Straps 81 and 82 when fastened together may encircle a user's left leg. Straps 83 and 84 when fastened together may encircle a user's right leg.

Leg harness 80, straps 81-84, buckles 86 and fastener members 88 and 89 may be identical or similar to their counterparts 50, 51-54, 56, 58 and 59, respectively. Additionally, the straps 81-84 may be made from, or could have over a portion thereof, hook and loop fasteners, such as VELCRO brand hook and loop material. This type of fastener would again allow adjustment while possibly avoiding the use of avoids the cumbersome buckles that may chafe the skin of, or make the user uncomfortable. Further, the hook and look-fastening material used for straps 81-84 also eliminates the need for fastener members 88 and 89.

Longitudinal strap (or lower torso strap) 70 extends upwardly from its first or lower end and passes through a transverse opening in base member 62 of clamp 60. Clamping member 64 of clamp 60 grips strap 70 to keep it from slipping. A user may allow the remaining portion of strip 70 to hang vertically downward if desired. However, the second (or upper) end of strap 70 may be provided with the second part 90 of a closure member, as for example, hooks of a hook-and-loop closure member. This enables a user to fasten the second (or free) end of strap 70 to the upper end of center web 30 in complimentary closure members 36 and 90, to avoid a rather lengthy dangling free end portion of strap 70.

FIGS. 6 and 7 illustrate a device 20 of this invention in use. A user puts of the device 20 so that the center web 30 is just behind the user's back above the waist and strap 70 hangs downwardly from the center back. The user then fastens the shoulder straps 51-54 and the leg straps 81-84 tightens these straps to fit, and pulls on the free portion of strap 70 (the portion between clamp 60 and the second end of strap 70) until the device 20 fits the user's height. The clamp 60 cooperates with the rough woven surface of strap 70 to permit a user to tighten (but not to loosen) the device 20. To loosen device 20, a user must open clamp 60 by pressing on the clamping member 64 of clamp 60 so as to release the strap 70.

When a user has put on the device 20 and properly adjusted it to his or her height and shoulder and thigh dimensions, the user will be restrained from bending the back to any appreciable extent. The elastic center web 30 offers considerable resistance to bending the back, and all straps 51-54, 70 and 81-84 cooperate with the center web to resist back bending since they are dimensionally stable. On the other hand, the user is free to bend at the knees in order to pick up an object. Health experts universally agree that bending the back may be harmful while bending at the knees is preferable.

The back brace of this invention offers a simple and effective device for preventing back strain while reaching down to pick up objects. This device is especially helpful in preventing injury from lifting heavy objects.

Dimensions are not critical. However, center web 30 is much wider than straps 51-54, 70 and 81-84 preferred embodiments. In an illustration and preferred embodiment, center web 30 may be 10 inches long and 3 inches wide, and each of the straps 51-54, 70 and 81-84 may be one inch wide.

A preferred embodiment has been shown and described in detail by way of illustration and not limitation. Variations and modifications can be made without departing from the scope and spirit of this invention.

What is claimed is:

1. A flexible back brace comprising:
a shoulder harness comprising;
a pair of first and second shoulder harness straps arranged at angles with respect to the brace's central axis, whereby each pair of first and second straps are adapted to encircle each user's shoulder and be connected to one another by first connection means;
a center web whereby one end of each of the pairs of first and second shoulder harness straps are radially connected;
a spring biased clamp that is radially attached to the center web in a longitudinally downward arrangement;
a leg harness comprising:
a pair of first and second leg harness straps arranged at angles with respect to the brace's central axis, whereby each straps are adapted to encircle each user's leg and be connected to one another by a second connection means;
a longitudinally extending strap oriented in a longitudinally upward arrangement, whereby one end of the pair of first and second leg harness are attached to the longitudinally extending strap in a radial arrangement, whereby the clamp of the shoulder harness receives the unattached end of the longitudinally oriented strap, thereby connecting the shoulder and leg harnesses.

2. A flexible back brace as recited in claim 1, further comprising a plurality of adjustable buckles located on each pair of first and second straps for accommodating a variety of chest and leg sizes of a user.

3. A flexible back brace as recited in claim 1, wherein the first connection means is comprised of a hook and loop closure member.

4. A flexible back brace as recited in claim 1, wherein said first connection means is comprised of a side lock fastener.

5. A flexible back brace as recited in claim 1, wherein the second connection means is comprised of a hook and loop closure member.

6. A flexible back brace as recited in claim 1, wherein the second connection means is comprised of a side lock fastener.

7. A flexible back brace as recited in claim 1, wherein the pair of first and second straps of the shoulder and leg harnesses have a width that is narrower than the width of the center web.

8. A flexible back brace as recited in claim 1, wherein the first straps of each pair of straps is arranged substantially horizontal and the second straps of each pair are arranged at an acute angle therewith.

9. A flexible back brace as recited in claim 8, wherein the angle between the brace's central axis and the second straps is about 30 degrees.

10. A flexible back brace as recited in claim 1, wherein the center web is comprised of a plurality of plies of elastic material.

11. A flexible back brace as recited in claim 1, further comprising a cloth reinforcing member that is attached to the lower end of the center web.

12. A flexible back brace as recited in claim 1, further comprising a cloth belt loop attached to the lower end of the center web, wherein the fixed end of the spring biased clamp is affixed.

13. A flexible back brace as recited in claim 1, wherein the straps of the shoulder harness and leg harness are formed from non-stretchable material.

14. A flexible back brace as recited in claim 1, wherein the longitudinally extending strap is formed from non-stretchable material.

15. A flexible back brace as recited in claim 1, further comprising:
a downwardly oriented longitudinal strap attached to the central region of the leg harness; and
a plurality of hook and loop material disposed on both the downwardly and upwardly oriented leg harness straps, such that the excess portion of the upwardly oriented strap which has passed through the spring biased clamp is held in position by the hook and loop material.

16. A method of bracing a user's back comprising the steps of:
centering a center web, containing a shoulder harness, behind a user's back;
fastening the shoulder harness around the user's shoulders;
fastening a leg harness around the user's legs;
connecting the shoulder harness to the leg harness by inserting an upwardly oriented non-stretchable strap, that is centrally attached to the leg harness, through a spring biased clamp attached to the center web; and
pulling the non-stretchable strap through the clamp until the user's back is sufficiently immobilized thereby restraining the user from bending over at the waist.

17. A method of bracing a user's back as recited in claim 16, further comprising the steps of:
attaching a downwardly oriented, non-stretchable strap centrally affixed to the leg harness; and
affixing the upwardly oriented portion of the non-stretchable strap via hook and loop material to the downwardly oriented strap.

18. A flexible back brace comprising:
a means for anchoring a plurality of straps around the shoulders of a user;
a means for anchoring a plurality of straps around the legs of a user;
a means for connecting the shoulder anchoring means and leg anchoring means together; and
a means for adjusting the length of the connection means to a length sufficient to restrain the user from being able to bend over at the waist.
